(19) United States
(12) Patent Application Publication
(10) Pub. No.: US 2020/0211680 A1
(43) Pub. Date: Jul. 2, 2020

(54) SYSTEMS AND METHODS FOR REMOTE CLINICAL TRIAL INTEGRATION AND EXECUTION

(71) Applicants: Tomasz Sablinski, Short Hills, NJ (US); William Douglas Clarke, Florence, MA (US); Alex Evan Greenberg, Brooklyn, NY (US)

(72) Inventors: Tomasz Sablinski, Short Hills, NJ (US); William Douglas Clarke, Florence, MA (US); Alex Evan Greenberg, Brooklyn, NY (US)

(21) Appl. No.: 16/722,478

(22) Filed: Dec. 20, 2019

Related U.S. Application Data
(60) Provisional application No. 62/785,092, filed on Dec. 26, 2018.

(51) Int. Cl. G16H 10/20; G06N 20/00; G16H 40/60

(52) U.S. Cl. CPC G16H 10/20; G16H 40/60; G06N 20/00

(57) ABSTRACT

According to another aspect, clinical trial execution can be improved based on a paradigm shift to a remote model. In various embodiments, the system manages and executes an asynchronous visit, that is a system based visit simulating doctor or clinician interaction for a respective clinical trial participant or a patient medical visit. According to various embodiments, the asynchronous visit enables patient or participant interaction with the system unbounded by time or place—this represents a leap forward in clinical trial execution, as both trained personnel availability and location restriction have significantly limited the ability to conduct trials. In some examples, these limitations have prevented qualified participants from engaging with a trial run. Thus the system avoids a variety of known problem with conventional trial execution, and for example, situations where a trial with a small qualifying population cannot afford to lose even one qualified patient.

### RELATED APPLICATIONS

This application claims priority under 35 U.S.C. § 119(e) to U.S. Provisional Application Ser. No. 62/785,092 entitled “SYSTEMS AND METHODS FOR REMOTE CLINICAL TRIAL INTEGRATION AND EXECUTION,” filed on Dec. 26, 2018, which application is incorporated herein by reference in its entirety.

### BACKGROUND

Conventional approaches for clinical trial execution face significant hurdles in their initial definition, subscribing participants, and during execution. For example, conventional trial approaches implement slow and time consuming patient enrollment processes. Typically, patient enrollment requires significant support and infrastructure, as enrollment is done at hospitals and/or through doctors. In some examples, the hospitals and/or doctors do not even focus on clinical trial development. Rather, they are engaging in trial development and execution as a sideline activity, while continuing the normal practice of seeing and treating patients.

It is further known that trial administration is complicated by appropriate selection of trial participants. Additionally, the actual execution of the trial is likewise hobbled by the inefficiency of requiring participants to come to clinical trial sites or selected medical facilities. These visits are typically required to capture participant data, take samples, administer treatment, among other options.

### SUMMARY

Various conventional approaches have attempted to simplify and streamline the burdens imposed on participants in clinical trial execution, but these conventional approaches simply have not made or incorporated the transition to a fully remote approach. The inventors have realized that there is a need for a remotely accessible platform that can be used to support a variety of clinical trial executions. According to some embodiments, the system can support multiple trial settings and manage remote trial execution with respective participants. According to one example, the system is configured to develop remote trial execution questions, identify follow up questions based on participant responses in real time by training artificial intelligence models to each respective trial being offered. In further examples, multiple AI models can be implemented for respective trials, where each is configured to handle aspects of participant interaction that would normally be handled by a clinician or physician, solicit and interpret participant responses to queries, and to guide participants in treatment administration and data or sample collection activities. Other embodiments execute rule based selections of questions, responses, and follow up in system driven interactive sessions with the participants.

According to various embodiments, the system is configured to train artificial intelligence models with a baseline set of clinical trial protocols, including queries for participants. In some embodiments, the system is configured to automatically identify interactive follow up queries based on received responses and present follow up queries in real time. The system can, in further embodiments, guide participants through sample collection and treatment administration needed for the trial. In some examples, the participants can request live advice on how to operate collection materials and the artificial intelligent models can respond with specific tailored directions to the respective participant with vocal instruction coupled with video based demonstrations. Feedback from the participants during the interactive sessions with the system can be used to refine instructions based models so that they are tuned (e.g., trained) to the individual participant, including for example, trained to preferences and/or capabilities of the individual. In further embodiments, the system can be configured to dynamically select from portions of available video responsive to analyzing patient questions and/or responses. For example, tailoring (e.g., editing dynamically) the delivered video to relevant portions and/or the portions needed to respond to participant question offers significant improvement both in the system (e.g., less bandwidth, processing, and memory is needed to deliver the targeted video or edited video portions) and efficacy of the execution (e.g., participants do not become overwhelmed or bored with volumes of irrelevant training video—a failure of many conventional approaches).

According to another aspect, clinical trial execution can be improved based on a paradigm shift to a remote model. Conventional approaches and execution require physical locations and doctor or clinicians availability to oversee treatment and/or sample collection. In various embodiments, the system manages and executes an asynchronous visit, that is a system based visit simulating doctor or clinician interaction for a respective clinical trial participant or a patient medical visit. According to various embodiments, the asynchronous visit enables patient or participant interaction with the system unbounded by time or place—this represents a leap forward in clinical trial execution, as both trained personnel availability and location restriction have significantly limited the ability to conduct trials. In some examples, these limitations have prevented qualified participants from engaging with a trial run. Thus the system avoids a variety of known problem with conventional trial execution, and for example, situations where a trial with a small qualifying population cannot afford to lose even one qualified patient.

In yet another aspect, a system mediated simulation of a doctor or clinical trial visit is offered as a tailorable service provided by the system. In various embodiments, clinical trial personnel can input the parameters of their respective trial into the system. The system can use pre-trained models in conjunction with newly acquired trial parameters (e.g., treatment specifications), patient data collection specifications (e.g., queries to be asked to patients), patient sample collection specification (e.g., biometric data capture, biological sample capture, etc.), to define and/or establish execution parameters for asynchronous visits that are tailored to the input trial parameters. In one example, the system determines which prior models could be used based on analysis of newly input trial parameters. The system can use supervised learning to identify patient questions that will yield quantitative responses aligned with trial execution requirements, as well as tailoring questions based on as feedback garnered during trial execution. In other examples, non-supervised learning models can be generated based on prior trial executions, prior questions, and participant responses. The non-supervised models can be executed to identify quantifiable questions and cluster those questions based on what the responses indicated in terms of a respective trial (e.g., what indicators are implicated, question and/or answer elicits an indicator of good prognosis or bad prognosis, question and/or answer identifies an improvement under treatment or worsening under treatment, etc.). In another example, the intelligent models can be configured to generate quantitative questions from previously executed trials. In further example, responses garnered from such questions can be input to intelligent models to provide follow up or focusing questions.

According to another aspect, the system can also monitor trial execution for anomaly detection (e.g., machine learning anomaly detection models can be trained to identify potential or predicted failure to adhere to clinical trial requirements, identify aberrant behavior, uncharacteristic operation parameters of therapeutic devices, sample collection devices, etc.). Past executions can be used to develop and/or refine compliance models that are used to predict noncompliant results and for example, predict trends towards non-compliance based on respective models. For example, trends in data collection (e.g., participant compliance with treatment and/or evaluation collection, various issues with sample collection, device operation, etc.) can be identified by the learning models before non-compliance or other issues occur. In some examples, the system can be configured to generate or request administrative amendments to protocol execution before such non-compliance occurs. According to various embodiments, the ability to predict and resolve non-compliance in trial execution represents functionality that various conventional systems cannot perform. This enhanced functionality provides significant benefits, and can in some examples salvage all the effort (e.g., trial definition, participant treatment and data collection, etc.) in a trial execution that would have been discarded for failure to meet trial requirements, through prediction and corrective action/recommendation, and/or by triggering intervention.

According to one aspect, an execution system is provided. The system comprises at least one processor operatively connected to a memory, a modelling component, executed by the at least one processor, configured to automatically identify queries based on a first machine learning model, automatically identify follow up queries responsive to the participants answers to the queries based on a second machine learning model, a visit simulator, executed by the at least one processor, configured to: present queries identified by the modelling component, accept responses from the clinical trial participant, present the follow up queries responsive to the participants answers in real time, and display instructions for treatment administration or data collection tailored to the participants requests.

According to one embodiment, the system further comprises a compliance evaluator component, executed by the at least one processor, configured to monitor participant compliance with client trial requirements and generate predictions for non-compliance in treatment and/or collection. According to one embodiment, visit simulator, executed by the at least one processor, is further configured to guide participants through use of measurement devices associated with a clinical trial execution.

According to one embodiment, the visit simulator is configured to automatically select audio and visual prompts to the participants. According to one embodiment, the visit simulator is configured to: monitor data capture from the measurement devices; generate a capture quality metric for the monitored data; and generate video and/or audio instruction to the participant to optimize localization, operation, or positioning of the measurement device to improve capture quality. According to one embodiment, the visit simulator is configured to: monitor video capture of the participant during use of the measurement devices; generate video and/or audio instruction to the participant to optimize localization, operation, or positioning of the measurement device to improve capture quality.

According to one embodiment, wherein instruction occurs in real or near real time. According to one embodiment, wherein the system is configured to generate an optimal data capture model for each respective participants, and generate tailored instructions to the respective participants based on identified positioning, localization, or operation for best data capture results.

According to one aspect, a clinical trial execution system is provided. The system comprises at least one processor operatively connected to a memory, a protocol analyzer component, executed by the at least one processor, configured to define protocol requirements to be performed by a plurality of clinical trial participants, validate pre-requisites for performing steps of the clinical trial protocol, define boundaries for time ranges to execute a virtual visit with a participant that meet at least a minimum temporal requirement defined by the protocol requirements, a visit simulator, executed by the at least one processor, configured to: present queries identified as at least part of the protocol requirements, accept responses from the clinical trial participant, display instructions for treatment administration or data collection based at least in part on the protocol requirements, and capture health or device data associated with the protocol requirements to be performed by the plurality of clinical trial participants.

According to one embodiment, the visit simulator is configured to generate and present follow up queries responsive to the participants answers in real time, wherein the follow up queries are tailored to the participants requests. According to one embodiment, the system further comprises a compliance evaluator component, executed by the at least one processor, configured to monitor participant compliance with client trial requirements. According to one embodiment, the visit simulator, executed by the at least one processor, is further configured to guide participants through use of measurement devices associated with a clinical trial execution.

According to one embodiment, the visit simulator is configured to automatically select audio and visual prompts to the participants. According to one embodiment, the visit simulator is configured to: monitor data capture from the measurement devices; generate a capture quality metric for the monitored data; and generate video and/or audio instruction to the participant to optimize localization, operation, or positioning of the measurement device to improve capture quality. According to one embodiment, the visit simulator is configured to: monitor video capture of the participant during use of the measurement devices; generate video and/or audio instruction to the participant to optimize localization, operation, or positioning of the measurement device to improve capture quality.

According to one aspect, a computer based method for clinical trial execution is provided. The method comprises defining, by at least one processor, protocol requirements to be performed by a plurality of clinical trial participants, validating, by the at least one processor, pre-requisites for performing steps of the clinical trial protocol, defining, by the at least one processor, boundaries for time ranges to execute a virtual visit with a participant that meet at least a minimum temporal requirement defined by the protocol requirements, presenting, by the at least one processor, queries identified as at least part of the protocol requirements; accepting, by the at least one processor, responses from the clinical trial participant; displaying, by the at least one processor, instructions for treatment administration or data collection based at least in part on the protocol requirements; and capturing, by the at least one processor, health or device data associated with the protocol requirements to be performed by the plurality of clinical trial participants.

According to one embodiment, the method further comprises generating and presenting follow up queries responsive to the participants answers in real time, wherein the follow up queries are tailored to the participants requests. According to one embodiment, the method further comprises monitoring participant compliance with client trial requirements. According to one embodiment, the method further comprises automatically guiding participants through use of measurement devices associated with a clinical trial execution. According to one embodiment, the method further comprises monitoring data capture from the measurement devices; generating a capture quality metric for the monitored data; and generating video and/or audio instruction to the participant to optimize localization, operation, or positioning of the measurement device to improve capture quality.

Still other aspects, embodiments, and advantages of these exemplary aspects and embodiments, are discussed in detail below. Any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

Various aspects of at least one embodiment are discussed herein with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. Where technical features in the figures, detailed description or any claim are followed by reference signs, the reference signs have been included for the sole purpose of increasing the intelligibility of the figures, detailed description, and/or claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. In the figures:

FIG. 1 illustrates a block diagram of an example execution system, according to one embodiment;

FIG. 2 is a concept diagram illustrating how asynchronous visits change conventional approaches to clinical trial execution, according to one embodiment;

FIG. 3 is a block diagram of an example special purpose computer system on which various aspects of the invention can be practiced according to one embodiment

FIG. 4 illustrates a block diagram of an example execution system, according to one embodiment;

FIG. 5 illustrates a block diagram of an example execution system, according to one embodiment;

FIG. 6 illustrates a block diagram of an example execution system, according to one embodiment;

FIG. 7 illustrates a block diagram of an example execution system, according to one embodiment;

FIG. 8 is a block diagram of system components and logic flow, according to one embodiment;

FIG. 9 is a block diagram of system components and logic flow, according to one embodiment;

FIG. 10 is a block diagram of system components and logic flow, according to one embodiment;

FIG. 11 is a block diagram of system components and logic flow, according to one embodiment;

FIG. 12 is a block diagram of system components and logic flow, according to one embodiment;

FIG. 13 is a block diagram of system components and logic flow, according to one embodiment; and

FIG. 14 is a block diagram of system components and logic flow, according to one embodiment.

### DETAILED DESCRIPTION

According to one aspect, an execution system is provided to manage asynchronous visits by patients or clinical trial participants. According to various embodiments, the system supports treatment and data collection unbounded by location or time. In some examples, the system can be configured to provide tailored support to multiple trial settings and manage remote trial execution with respective participants. According to one example, the system is configured to develop remote trial execution questions, process participant responses, identify follow up questions based on responses in real time, and advise on treatment execution and/or data gathering with automated tools and/or learning models. In further embodiments, learning models can evaluate trial compliance (e.g., conformity to treatment administration, compliance with participant execution, etc.).

According to some embodiments, one or more intelligent models are executed for each respective trial being offered. The multiple AI models can be implemented for respective trials, where each is configured to handle aspects of participant interaction that would normally be handled by a clinician, nurse or physician (e.g., solicit and interpret participant responses to queries, and to guide participants in treatment administration and data or sample collection activities, among other options, request feedback on treatment/collection burden, and likelihood to fulfil course of treatment, etc.).

Examples of the methods, devices, and systems discussed herein are not limited in application to the details of construction and the arrangement of components set forth in the following description or illustrated in the accompanying drawings. The methods and systems are capable of implementation in other embodiments and of being practiced or of being carried out in various ways. Examples of specific implementations are provided herein for illustrative purposes only and are not intended to be limiting. In particular, acts, components, elements and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

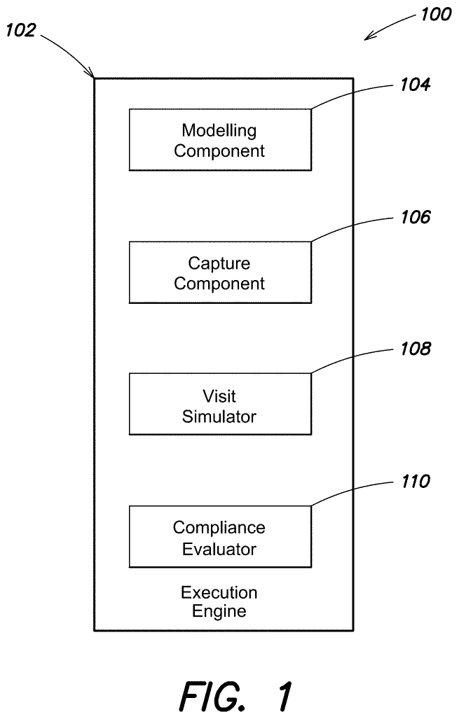

FIG. 1 is an example block diagram of an execution system 100. The execution system 100 is configured to manage and execute asynchronous visits to guide treatment administration and treatment efficacy data collection. The system 100 is further configured to provide asynchronous visits as a service to which various clinical trial personnel can engage to execute their own trial under the asynchronous visit model. As discussed, the asynchronous visit model provides automatic data collection and interaction (e.g., automatic identification of issues, targeted questions to address issues, and series of follow up questions tailored specifically to participant responses, among other options). According to some embodiments, the system 100 can be a stand-alone system executing the disclosed functionality or may instantiate an execution engine 102 configured to execute the discuss functions, algorithms, and/or logic. In yet other embodiments, the execution system 100 and/or engine 102 can be a cloud based system that services requests and provides interactive functionality to hosts of participants and multitude of clinical trials.

According to one embodiment, system 100 can include a modelling component 104 configured to build tailored execution models for each respective clinical trial. In one example, the system provides pre-trained model(s) including a set of questions which can be used in conjunction with details collected on a respective clinical trial to establish a learning model executed to present initial groups of questions to clinical trial participants during an asynchronous visit (including, for example, treatment administration, data/sample collection, etc.), analyze responses, and to select further questions to present, for example, until the analyzed responses are indicative of qualitative measures for the respective trial (including for example, stable condition, improving condition, worsening condition, etc.). In some embodiments, the system can include a capture component 106 configured to ingest or accept information from clinical trial personnel that defines the scope and requirements for executing a clinical trial (e.g., population qualifiers, registration requirements, end-points, indicators, devices required, questions to address with patients, etc.). The system can process the input information against pre-trained models to generate tailored execution models for the respective trial. In other embodiments, supervised learning is used to establish a functional model for presenting questions to participants, and or guiding treatment/data collection sessions automatically.

In various embodiments, the modelling component is configured to support dynamic interactions with a participant during a treatment/data collection session that are executed by a visit simulator component. In various examples, the system automatically executes asynchronous visits to manage treatment and/or data collection.

According one embodiment, the system 100 can include a visit simulator component 108 configured to dynamically and automatically interact with clinical trial participant. According to one embodiment, the simulator 108 provides dynamic questions and responses based on a learning intelligence model. In other examples, the simulator can process a series of expert guide rules to select questions and provide responses to the clinical trial participant dynamically.

In further embodiments, the simulator 108 is configured to display visual guides in the form of video segments and communicated audio signals, that provide responses to participants in real time. In one example, the learning model is executed to select or edit video segments so only a relevant portion of video is communicated and displayed to a participant, that is relevant and responsive to the participant's request. In one example, the execution system 100 and/or simulator component 108 is configured to display video instruction tailored to the received question “how do I collect my treatment data” or “how do I operate (supplied device).” According to one embodiment, the system uses artificial intelligence to interpret the question and select the appropriate response. In one example, the intelligent model identifies a client question based on analyzing received audio, which can include: “how do I” and a device name to select appropriate response and/or tailor portions of existing video for display to the participant.

In some examples, the visit simulator component 108 can include video analytics to capture and analyze video of a participant administering treatment or capturing their data via a supplied device to determine the participant is performing the operations correctly. Deviations in execution can be detected by the system, triggering instructive video and/or audio segments for presentation at the participant's device.

According to further embodiments, the system can include a compliance component 110. The compliance component is configured to determined is trial protocol requirements (e.g., data collection and/or treatment administration, etc.) are being followed. For example, a trail may specify treatment and monitoring/data capture must occur week or bi-weekly (and may include a variance window of +/−one day). The system can monitor participant compliance with those requirements. Based on the monitored data, the system is configured to predict candidate for non-compliance. If the population of candidates for non-compliance grows to a threshold size or increase at a threshold rate, the system can automatically take corrective action—e.g., send reminders, trigger asynchronous visit sessions, notify clinical trial administration, etc.) In further examples, the system automatically (e.g., based on a learning model or rule based execution) introduces additional questions on the burden associated with treatment and/or data collection. Based on responses or if the increase in candidate for non-compliance is large, the system can generate and automatically propose administrative amendments to the clinical trial protocol. Such administrative amendments can be limited to timing of treatment, timing of data collection, etc. In various embodiments, the system is configured to generate these types of amendments that can reduce the burden of trial participants and potentially save the trial execution from non-compliance.

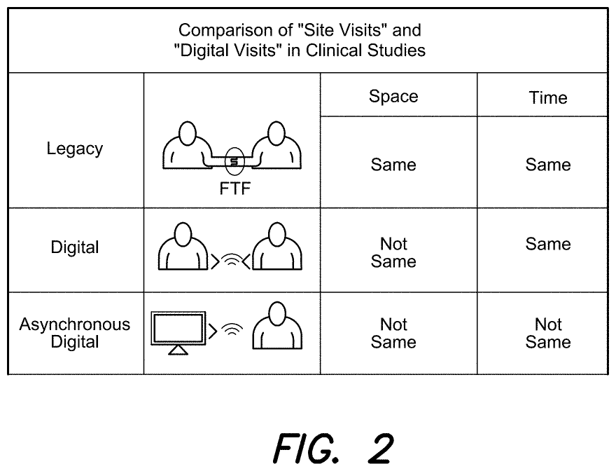

FIG. 2 is a concept diagram illustrating a logical element of the execution system, the asynchronous visit and how the asynchronous visit changes conventional approaches to clinical trial execution. According to various embodiments, the asynchronous visit divorces clinical trial execution from legacy requirement of having a participant and a clinician or physician in the same place at the same time. Additionally, the asynchronous visit improves over conventional attempts at remote access administration. For example, some conventional approaches resolve the issue of needing a hospital or office site to provide medical advice or administer some treatments. However, these approaches are limited in the time that they can be executed. In particular, these approaches still require that a clinician or physician attend a remote session at the same time as the participant provide advice or monitor participant activity.

As discussed above with regard to FIG. 1, various aspects and functions described herein may be implemented as specialized hardware or software components executing in one or more special purposed computer systems. In some examples, various aspects and functions may be distributed among one or more computer systems configured to provide a service to one or more client computers, or to perform an overall task as part of a distributed system. Additionally, aspects may be performed on a client-server or multi-tier system that includes components distributed among one or more server systems that perform various functions.

According to various embodiments, conventional computer systems can be improved based on the present disclosure, in some examples, enabling new functionality unavailable in various conventional systems, in other examples, by improving execution efficiency (e.g., reducing memory required, improving accuracy of interactions, reducing network traffic, capturing and/or communication limited (e.g., only) relevant video instruction, etc.), among other options.

In various embodiments, the system can also be engaged by clinical trial personnel to build and deliver all the equipment, medication, etc. needed for a participant to engage in a trial remotely. In one example, each participant receives a specially configured computing device (e.g., laptop, tablet, etc.) tailored for the participant, measurement devices (e.g., blood tester, heart rate monitor, EKG sensor system, cameras, respiration monitors, oxygen monitors, blood pressure devices, etc.) and intelligent guides on the use of respective devices. In one example, the system can execute a remote session that provide visual guides (e.g., tailored video) on the use and capture of data using respective measurement devices. For instance, video monitoring of the patient can be processed by the system, which is configured to identify better use of the measurement device. In some embodiments, the system is configured to dynamically generate (e.g., edit or create) video advising the participant on better use of the device (e.g., how to hold, capture, etc.), video on better placement of the device for measurement, etc. In conjunction and or separately from video based analysis, the system can monitor captured data to provide dynamic and in some cases immediate feedback on quality of the data reading being captured by a measurement device.

In one embodiment, as a participant is instructed to move the position of a measurement device using video based instruction automatically generated by the system, the system can monitor a capture quality (e.g., data signal strength, data capture rate, noise or interference information, etc.) on data being received from a measurement device and provide visual feedback on an increase or decrease in a quality metric (e.g., improved or decreased signal strength, among other options) to the participant. The system can employ capture quality (e.g., based on analysis data received from the device, interference detected by the device, etc.) and video based instruction to have the participant localize the best positioning and/or the best location on their person and/or at their location for using measurement device sensor systems.

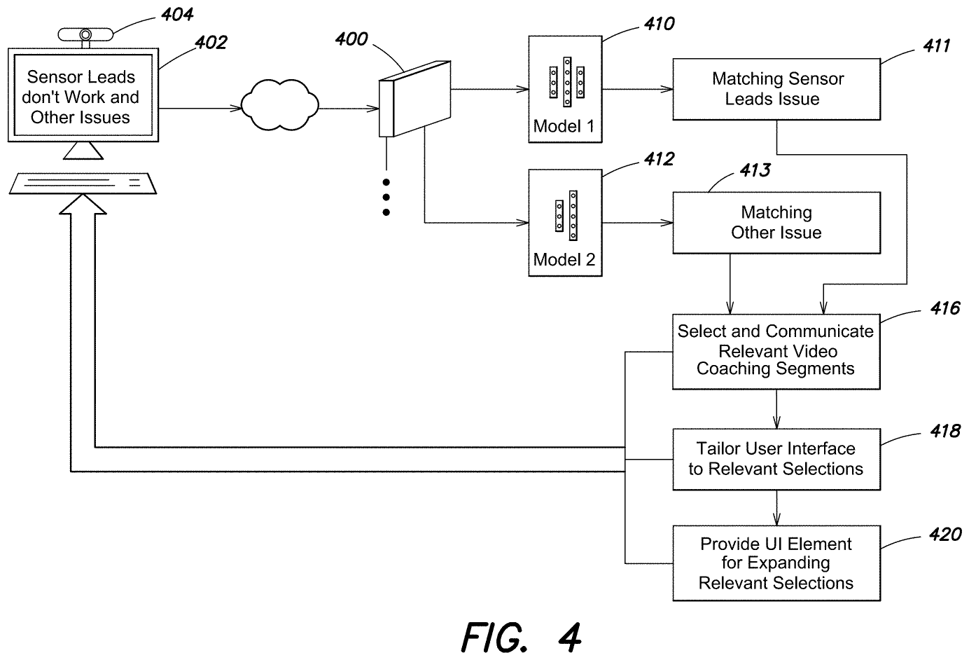

FIG. 4 is a block diagram of an execution system 400. According to some embodiments, an end user can interact with system 400 from a remote location at their own computing device 402. The user's computer device can include audio and video capture components (e.g., 404). In some embodiments, the user may sign onto system 400 to execute a data collection session or a scheduled clinical trial remote session. As part of the clinical trial protocol, users can be given ranges of dates and time over which to complete specific actions. In one example, the system monitors and reminds users to login and execute the steps of their respective protocols. In various examples, by providing date and time ranges, user compliance is vastly increased, and the system provides significant flexibility to the user to perform clinical trial protocols in the comfort of any remote location they choose.

In some embodiments, the user may encounter issues with the clinical trial protocol, data collection devices, or any part of a therapeutic regimen that is part of the clinical trial. In some embodiments, the system can use live video data (e.g. captured by 404) to identify issues automatically. According to one example, the system can analyze live video feeds to determine if a user is using a clinical trial device appropriately and/or following the steps of the protocol. If not, the system can automatically identify relevant material to send to the user to resolve the issue. For example, misuse or improper use of the device can lead to bad data or sample capture. The system can identify that the user is not using the device correctly based on analysis of captured video. In some examples, the system can identify issues based on analysis of captured audio alone or in conjunction with captured video. For example, user exclamation during execution is indicative of issues and in conjunction with video analysis can yield information on the potential issue. The various data inputs can be used by machine learning models to match those inputs to specific issues (e.g., at 411 “matching sensor issue” or 413 matching other issue).

In one example, a user can provide information to the system “no data captured” as a text or voice input, and the system can analyze any audio or video during the remote section to determine if user error, device error, etc., may be the cause of the problem. According to one embodiment, an intelligent model can accept audio and video input as well as user free text information and match that data to issues associated with the user, the device being employed by the user, and/or regimen issues.

In one example, the system can identify that the user is employing the wrong device during data collection, and/or that the user is missing devices for a particular set of steps. In one alternative example, the system can identify improper handling and/or sterilization techniques. To provide another example, the system can analyze captured video to determine that the user failed to apply a sensor gel (e.g., sensor gel not present in video, or simply was not used, etc.) to data collection pads, which may be the source of a “no data” capture error.

Responsive to matching the user input and/or captured data to a potential issue, the system can identify management information and/or tools that can help the user resolve the issue. In one example, the system can provide instructional video tailored specifically to the issue identified by the machine learning model. In some settings, the system captures segments of training video that are specifically directed to the issue user is having from longer training video files. The system communicates the tailored training video that is relevant to the issue rather than sending a link to the training video or communicating the entire video. In some examples, the system can be configured to begin play on the relevant sections automatically. In some examples, the system is configured to dynamically adjust the user interface displayed to the end user to accommodate the selected training video and may also include use diagrams that are specific to the user's issue to facilitate proper use and/or data capture.

According to one embodiment, system 400 can include intelligent models for each device being used during a clinical trial. For example, model 410 can except video and audio and user free text input and output matches to potential issues with the respective device. At 416 the system can select and communicate relevant video coaching segments and trigger their playback at the end user device 402. As discussed, system 400 can tailor the user interface dynamically to provide the most relevant information (418) in a prioritized area of the user interface and helpful but potentially less relevant information in de-emphasized portions of the user interface. In further embodiments, system 400 can tailor the user interface to include additional UI elements (420) for expanding relevant selections, integrating additional content (e.g. coaching video, etc.), and UI elements for requesting human intervention or assistance.

According to various embodiments, each clinical trial and/or clinical trial protocol, and/or clinical trial regimen can include a plurality of intelligent models (e.g., 410, 412, and/or any number of additional models indicated by “ . . . ”) trained on prior executions and/or usage of the same or similar devices being used in a clinical trial or clinical trial protocol. The various models can provide probabilistic matching to issues encountered in prior executions and the matching can be used to select resolution information.

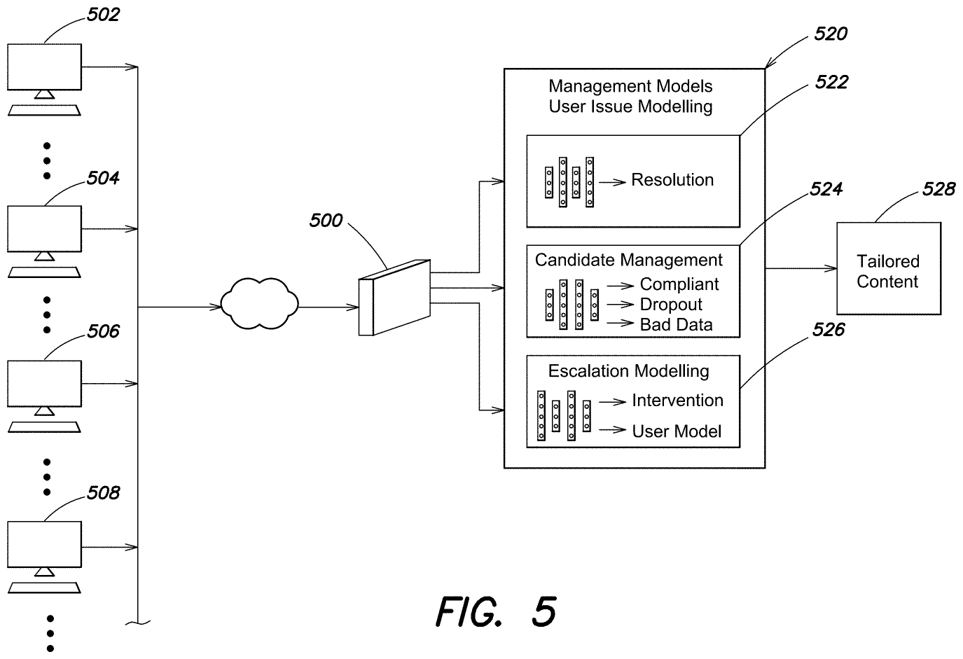

FIG. 5 is a block diagram of an example environment, including an execution system 500. The system 500 can be accessed by any number of remote users (e.g., 502-508, with . . . showing additional users) who are participating in a clinical trial. In some examples, each user can be issued the devices needed to collect samples and/or follow the clinical trial protocol. The users are given time windows in which to log into the system and perform a remote clinical trial visit. In various embodiments, the system 500 can manage the execution of the remote visit. For example, the system can include a number of intelligent models to facilitate remote execution of a clinical trial article.

In one example, the intelligent models (e.g., at 520) can include user issue modeling (e.g., 522) and mappings to potential resolutions. In another example, the system can include intelligent models to determine which users are compliant with the clinical trial protocol (e.g., 524). The compliance models can include predictors for compliant end-users, noncompliant end-users, and users likely to generate bad data during collection or execution of the clinical trial protocol. In various embodiments, the system 500 can take a variety of remedial actions responsive to predictions of noncompliance, prediction of drop out, prediction of bad data collection, among other options. In one example, indicators of bad data collection triggers coaching video, audio, diagrams, etc.

In other embodiments, data collected during a remote session can be analyzed by escalation models (e.g., 526). The escalation models are configured to determine if human intervention should be engaged based on data collected over the course of the user's participation. In some embodiments, the intelligent modeling has been trained on execution of similar or historic protocols and can analyze incoming data to determine if severe health related issues may or are likely to result. Responsive to such indicators, the system can trigger intervention protocols.

According to various embodiments, the system is configured to analyze data inputs captured during remote sessions and over the course of all remote sessions. The system can use the captured data as inputs to machine learning models that can classify the importance and/or match the inputs to potential issues. Once classified or matched the system can provide tailored content (e.g., 528) specific to the classified or matched issue.

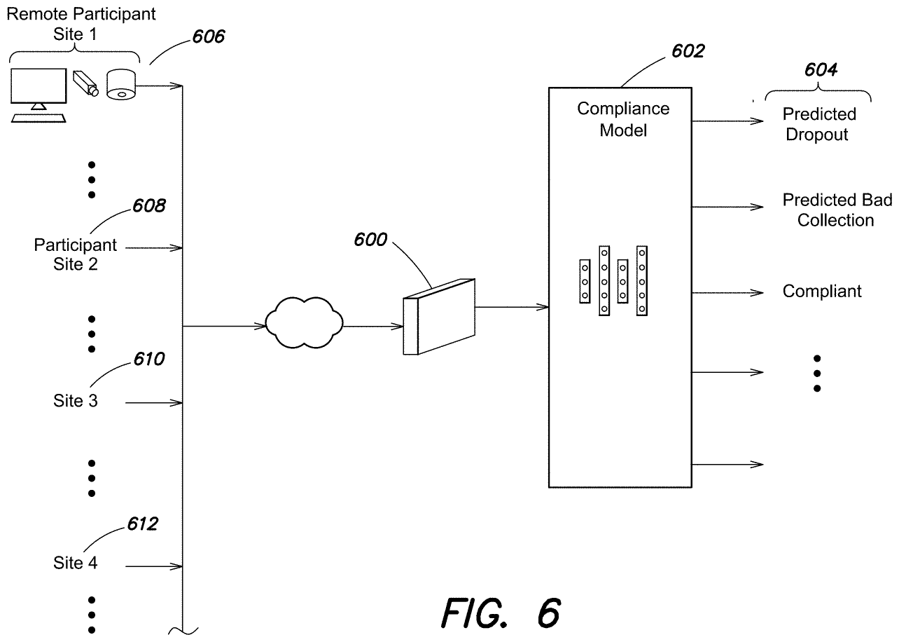

FIG. 6 shows a block diagram of an example execution system 600 and an intelligent model 602. In this example, the intelligent model is configured to analyze remote session data from a plurality of user locations (e.g., 606-612) to determine user compliance with the clinical trial protocol. For example, the compliance model is configured to generate predictions (e.g., at 604) on whether respective users may drop out of the clinical trial protocol, predict when users will make bad data collections, and or predict when the user is going to be compliant with the clinical trial protocol. Other predictions can be made regarding user compliance, potential for bad data collection, likelihood of compliance, and other classifications.

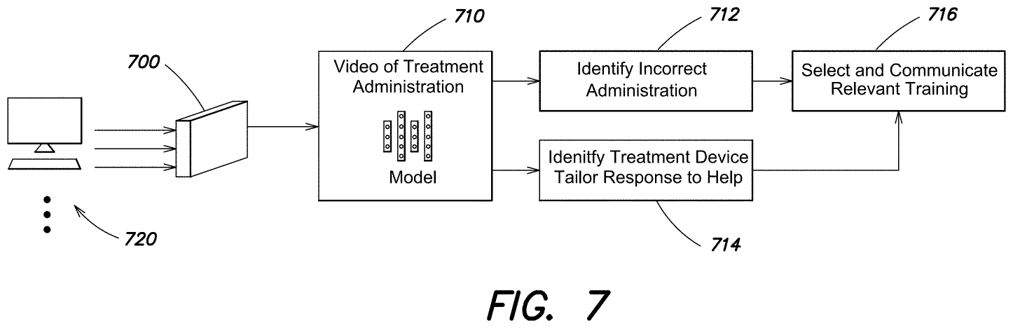

FIG. 7 shows a block diagram of an example execution system 700. System 700 can include an intelligent model configured to analyze video and/or audio input 710 during treatment administration to determine any issues, including incorrect administration 712 and issues with treatment devices 714 or regimen. Responsive to identifying such issues, the system can select and communicate relevant training material at 716 to a remote user or user population 720. As part of selection of the relevant training material, the system can be configured to capture segments of training video specific to an identified issue or problem. In such settings, the system is configured to communicate only the material needed and not overwhelm the user with an entire training video where specific segments of the video would resolve the user's issue.

According to one embodiment, a machine learning model can be trained on video observations of proper use of various devices employed in respective clinical trials. The trained model can analyze video feeds from user remoter sessions and determine if the end user is employing their device properly. In further examples, the model can provide outputs to identify improper use with reference to timing of the error or an associated function. The system can use the identification of improper use and the associated timing/function to select various remedial actions or communications. For example, alert messages can be shown to the user during a remote session, and training video demonstrating proper user for the identified function/timing can be captured and communicated to the end user. In yet other examples, the system can override the normal user interface display to provide the alert and/or automatically play the training video.

In further embodiments, additional machine learning modals can be executed to identify and/or predict users who are likely to drop out of a clinical trial protocol, or who are likely to fail to meet the steps for a clinical trial protocol. In some embodiments, machine learning models are trained against historical trial executions and information associated with the trial participants. The models are tuned to identify participation behaviors and participant characteristic indicative of improper compliance with trial regimens and/or indicative of participants who drop out of various trials. The trained model can be executed on the system to predict based on end user information and remote session, which user display behaviors indicative of dropping out. In further embodiments, the system can take remedial action, for example, in response to a drop out probability becoming more likely than not. In some examples, the system can tailor the remedial action to the user, requesting coaching sessions to link clinical personnel to the user during their remote session, request exceptions in the trial protocol to resolve user issues, etc. In such settings, the system can actively maintain participants in the trial execution, where conventional approaches simply oversubscribe to account for drop out.

### Virtual Visits Examples

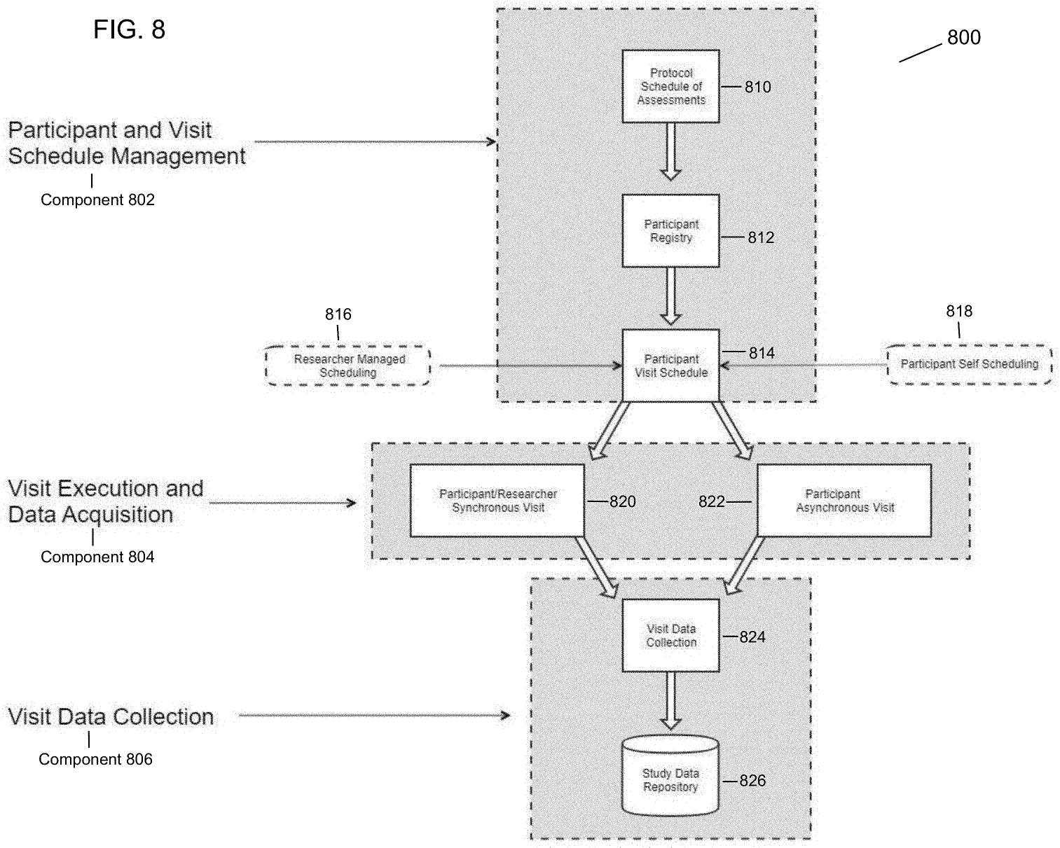

According to some embodiments, the execution system be implemented as a transparent virtual trial system (“TVT”). Various embodiments of the system are configured to break a clinical trial protocol down into constituent parts, allowing the system to schedule smaller “visits” which can be executed asynchronously by patients. The system enables the user to access the researcher portal to configure the trial protocol for virtual execution. According to one embodiment, the system enables a fully virtual trial execution platform. FIG. 8 is a block diagram of an execution system and logic flow 800.

According to some embodiments, the execution system can include a plurality of components. In one embodiment, the execution system includes a participant and visit schedule management component 802. The schedule management component can be configured to analyze schedule of assessments defined by clinical trial protocol and establish a participant visit schedule for the participants to find in a participant registry 812.

In some embodiments, the system can facilitate researcher managed scheduling at 816. In other examples, the system can facilitate participant self-scheduling at 818. In other examples, the system can derive scheduling ranges from a schedule of assessments defined by protocol (e.g. 810). Those scheduling ranges can be made available to participants defined in the registry (e.g. 812).

In some embodiments, once user input has been received or researcher managed scheduling has occurred, a visit execution and data-acquisition component 804 can operate to collect data for clinical trial protocol. For example, a participant/researcher synchronous visit (e.g. 820) can be executed by the system. In the synchronous visit example, a participant and researcher participate in an assessment at the same time but not necessarily in the same location. In further example, system intelligent models can take the place of the researcher, and execution of the visit can be managed by the system at any time, and any place desired by the participant with system-based coaching. In another example, if an asynchronous visit may be executed (e.g. 822). In an asynchronous visit the participant is able to conduct their own assessment without participation by a researcher and/or the system. In some examples, even for asynchronous visits system based help and intelligent models can provide assistance.

According to one embodiment, a visit data collection component 806 can be configured to collect any data at 824 from participant visits (e.g. synchronous and/or asynchronous). In various examples, the system can be configured to process and/or aggregate data to generate a study and data repository 826.

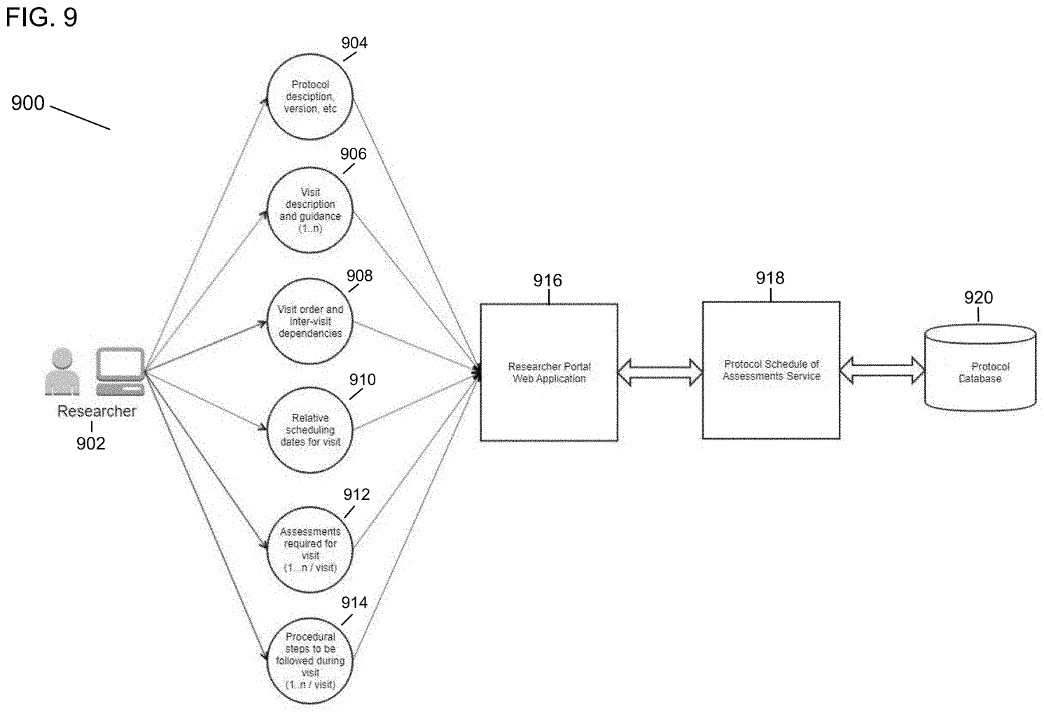

Shown in FIG. 9 is a block diagram of system components and process flow 900. According to various embodiments, the system facilitates defining remote “visits” which can be executed asynchronously by patients. The system enables users to access a web portal to configure a trial protocol for virtual execution.

According to one embodiment, a researcher (e.g. 902) can access a researcher portal web application 916. The researcher can enter information on a number of aspects of a clinical trial protocol. For example, the researcher can enter information on protocol description, protocol version, etc. (e.g. at 904). In another example, the researcher can enter information on visit description and guidance for any number of visits (e.g. at 906), visit ordering and enter any visit dependencies (e.g. 908), relative scheduling dates for conducting virtual visits (e.g. 910), assessment required for one or more visits over any number of visits defined in the protocol (e.g. 912), and procedural steps to be followed during a visit for any number of visits (e.g. 914), among other options. In various embodiments, the researcher and/or the system can use the aggregated information to define a protocol schedule of assessments and or services that must be performed. Once defined the information and schedule can be saved as part of a protocol database 920.

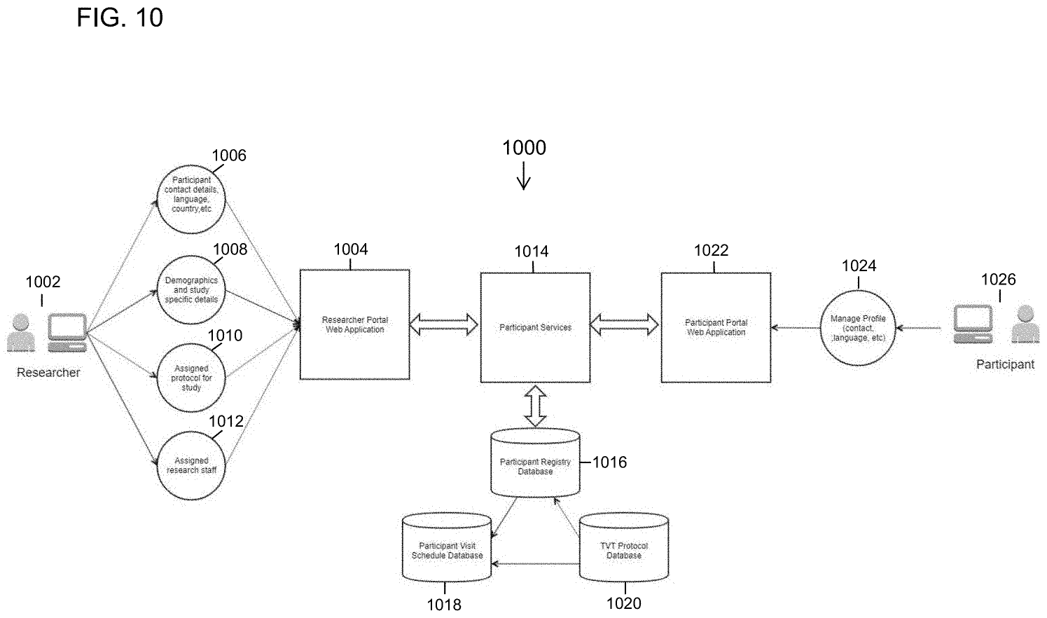

FIG. 10 is another block diagram of system components and process flow 1000. According to one embodiment, system users (e.g., researchers or other study staff) enter participant metadata into the system and assign each participant to a protocol. The system can also establish a “schedule” for the participant based on their assigned protocol. The schedule is used by the system to drive reminders send to researchers and participants.

According to one embodiment, the researcher 1002 can access a researcher portal application (e.g., 1004). The researcher can enter various metadata to define information for clinical trial execution. In one example, the researcher can define participant contact details, language, country, etc. at 1006. In another example, the researcher can enter information on demographics and study specific details at 1008, an assigned protocol for study at 1010, and information on assigned research staff at 1012, among other options.

According to one embodiment, the information entered in the researcher portal can be used by a participant services component 1014 to define information on participants in a registry database at 1016, which may include or communicate with a participant visit schedule database 1018 and/or a protocol database 1020. In further embodiments, participants may also access the system via a respective interface (e.g. 1022). In one embodiment, participant 1026 is able to manage their profile information 1024 via the portal.

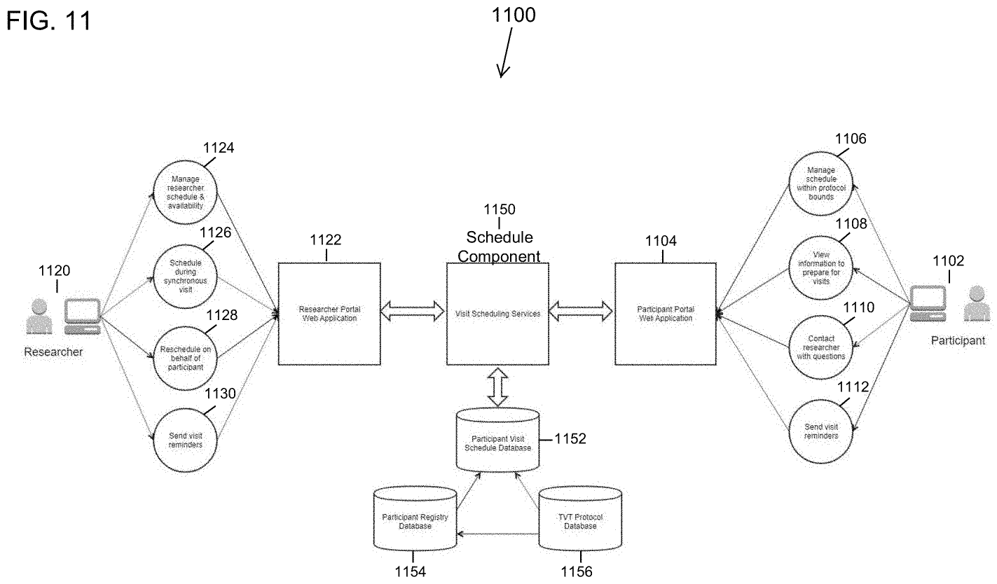

FIG. 11 is a block diagram of execution system components and process flow 1000, according to one embodiment. In various embodiments, the system facilitates both researcher and participant functions. For example, a participant is provided with guidance about when appointment must be scheduled in order to be compliant with the study protocol. The system can include a schedule component configured to display an easy to use interface for a participant to self-schedule their appointments. In another example, the system can display another interface configured to allow researchers to schedule an appointment with participants. Various operations can be executed during a virtual visit with the participant or may be executed as part of ad-hoc visits when the researcher needs to make contact with a participant. In various embodiments, system logic and/or intelligent models discussed herein can replace the operations executed by the researchers.

According to one embodiment, a researcher 1120 and/or a participant 1102 can access the system to perform various virtual trial operations. According to one example, a participant 1102 can access the system via a participant portal 1104 to manage their schedule (within system defined protocol boundaries) at 1106. In other examples, the participant can access the system to view information to prepare for visits at 1108, contact researchers with questions at 1110, and send or receive visit reminders at 1112, among other options.

In some embodiments, the researcher 1120 can access the system via a researcher portal 1122 to manage researcher schedule and availability at 1124. In other examples, a researcher can access the system to schedule synchronous visits at 1126, reschedule virtual trial sections at 1128, and communicate or receive visit reminders at 1130, among other options.

According to one embodiment, the information input by researchers and/or participants can be used by a schedule component 1150. The schedule component can be configured to reconcile availability requested times and resolve conflicts and schedules while preserving the requirements of a clinical trial protocol. In some examples, the system maintains this information in the participant visit schedule database 11 52. The schedule database can be in communication with a participant registry database 1154 and/or and protocol database 1156, among other options.

In various embodiments, the system is configured to analyze the defined participant schedule to drive virtual visits (e.g., between researchers and participants or the system). According to one embodiment, this is a part of creating a fully virtual visit. The researcher and the participant are in different spaces but in the same time. This allows the participant to execute visits from home, from their office or any other private location.

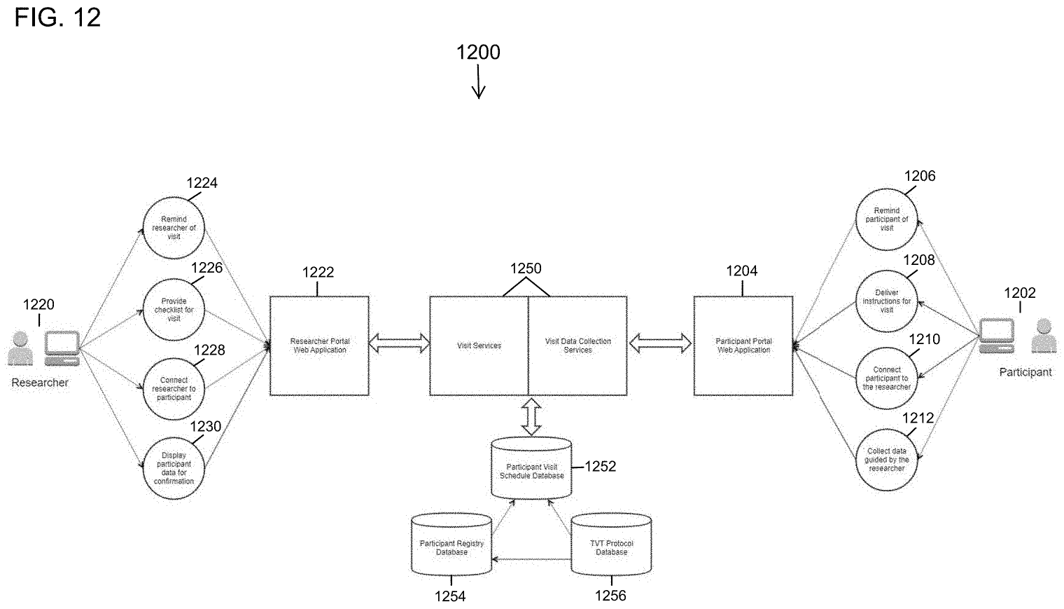

FIG. 12 is a block diagram of an execution system and logic flow 1200. According to one embodiment, a participant 1202 can access the system via the participant portal 1204 to receive reminders of visit sessions at 1206. In other examples, the system can be configured to deliver instructions for a visit at 1208, connect the participant to a researcher at 1210, and collect data during a session execution (for example, guided by the researcher or the system), among other options.

In other embodiments, a researcher 1220 can access a researcher portal 1222 to communicate or receive reminders of scheduled visits at 1224. In some examples, a researcher can also access the system to provide checklists for a visit at 1226, connect a researcher to a participant at 1228, and display participant data for confirmation at 1230, among other options. The data input by the respective users can be used by a visit execution component 1250. The visit execution component can be configured to manage data collection, provide coaching and/or training information for completing a virtual visit, and monitor participant actions to determine compliance or to provide assistance. In various embodiments, the data collected during a virtual visit can be stored in a participant visit schedule database 1252 and/or a participant registry database and/or a protocol database.

According to some embodiments, the system can manage execution of participant-only virtual visits in which case the researcher, and the participant are separated by both space and time. The participant will follow the direction (e.g., system prompts) in the participant portal and execute the appropriate steps required for their visit. In some embodiments, the system is configured to notify a researcher that the participant has completed their visit and can view the captured data on their own schedule. In other embodiments, the system is configured to monitor the participant as they execute their virtual visit, provide feedback, resolve issues, and/or automatically deliver relevant training material, among other options.

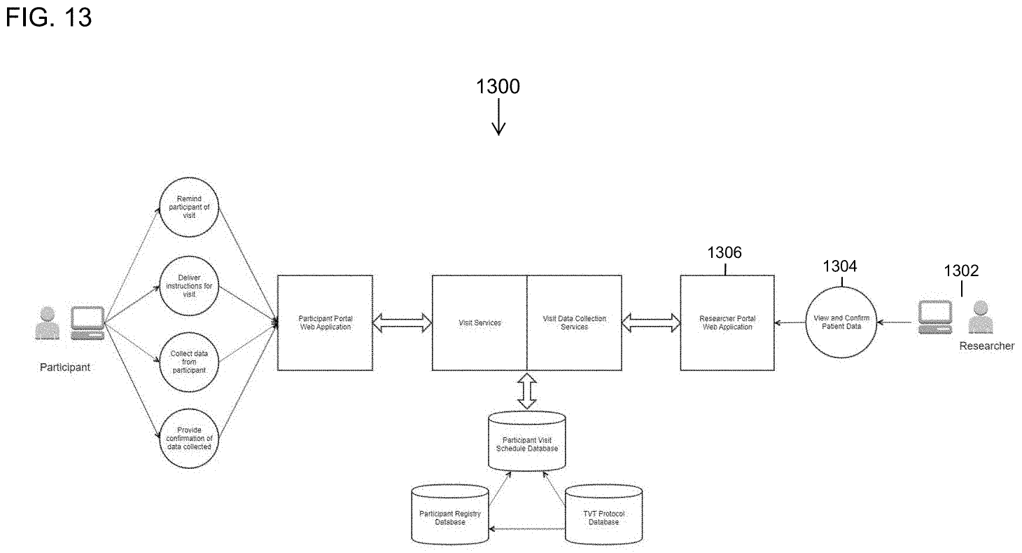

FIG. 13 is a block diagram of an execution is system and logic flow 1300. For purposes of clarity the additional functionality of this embodiment is emphasized. For example, a researcher 1302 can access a researcher portal 1306 to view and confirm patient data submitted during a virtual session. In other embodiments, the system can monitor the execution of the virtual visit and data collection. In further example, the system can monitor use of data collection devices and identify and resolve issues associated with data collection and/or execution of any step of the clinical trial protocol.

According to various embodiments, the data collected by the system is collected either through the participant portal or can be collected via a third party partner system. In one embodiment, the collected data is delivered to the system and can be made available through the researcher portal (e.g., for review by the researcher). In some embodiments, the collection, cleaning and validation of the participant data can be executed on the system before results are used. In other embodiments, third party data processing can be invoked to clean and validate the virtual visit data.

In some embodiments, the system is configured to validate that a participant is in position of the needed devices and/or therapeutic agents to perform and steps required during a virtual visit. In some examples, the system can validate the presence of devices or medicine via capturing and confirming RFID signals. In other examples, the system can be paired with the devices and communicate with them directly to determine location and/or presence during a virtual visit. Video analysis can also be employed to validate devices needed and/or medication to administer, among other options.

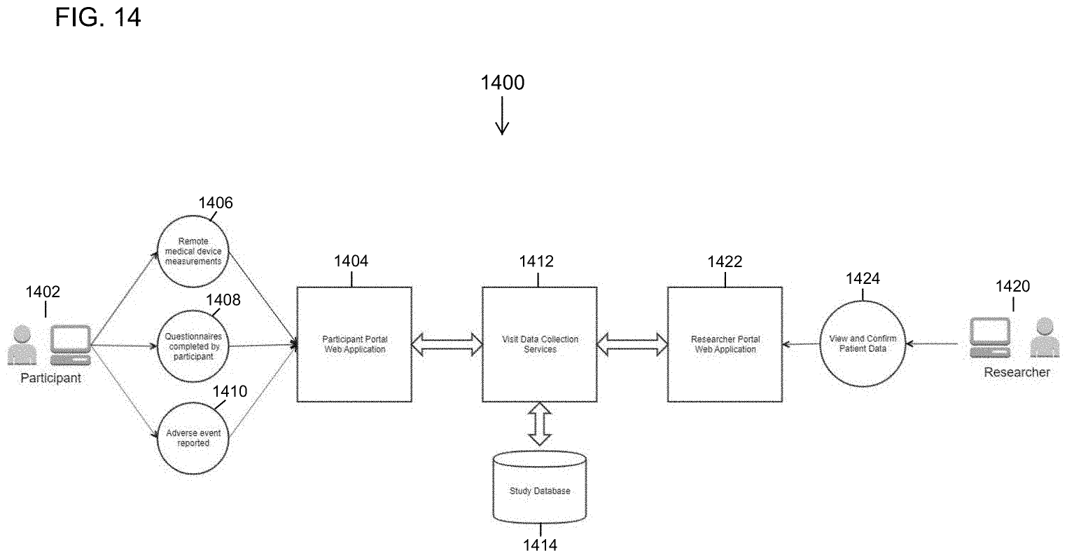

FIG. 14 is a block diagram of an execution system and logic flow 1400. At 1402, a participant can access the system via a participant portal 1404 to execute a virtual session. During the virtual session, the participant can use and collect data from remote medical devices, and the measurements obtained can be sent to the system. In other examples, questionnaires will be completed by a participant during the virtual visit at 1408. In some examples, the system can select the questionnaires that should be completed by the participant. In yet other examples, the system can dynamically adjust the questions to be asked and answered based on intelligent modeling and/or rule-based selections of questions and needed responses. In yet other examples, a participant can use the portal to report adverse events to the system at 1410.

In some embodiments, the system can include a data collection component 1412 configured to manage collection capture of participant data during a virtual visit. The resulting data can be stored in a study database at 1414. In some embodiments, a researcher 1420 can access the system via researcher portal 1422 to view and confirm collected patient data. In some examples, the researcher 1420 is responsible to verify the data collected, and to determine that the data was appropriately captured. In other embodiments, the system can provide monitoring and validation of capture data. In one example, the system can monitor data collection by a participant in determining whether or not the participant is appropriately using the devices and appropriately following the instructions of the clinical trial protocol. If the user does not appropriately use the devices and/or follow protocols, the system can automatically deliver coaching information to correct the user behavior. In further example, the system can display morning messages and use diagrams, and/or alerts researchers or other clinical trial administrators if a deviation poses health safety risks.

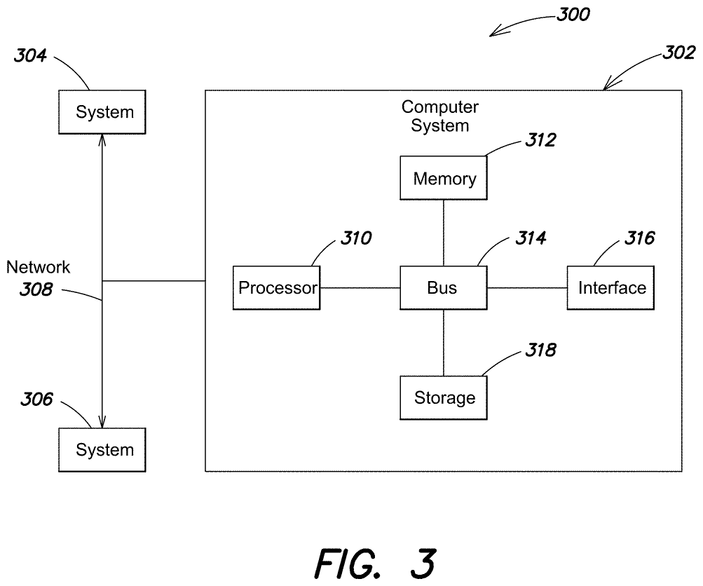

Referring to FIG. 3, there is illustrated a block diagram of a distributed computer system 300, in which various aspects and functions are practiced. As shown, the distributed computer system 300 includes one more computer systems that exchange information. More specifically, the distributed computer system 300 includes computer systems 302, 304 and 306. As shown, the computer systems 302, 304 and 306 are interconnected by, and may exchange data through, a communication network 308. For example, an execution system and/or engine can be implemented on 302, which can communicate with, for example, a simulation component on 304, and/or other systems implemented on 306 (e.g., capture component, etc.), which can operate together to provide the execution system functions as discussed herein. In other embodiments, execution system can be implemented on 302 or be distributed between 302-406.

According to various embodiments, conventional computer systems can be improved based on the present disclosure, in some examples, enabling new functionality unavailable in various conventional systems, in other examples, by improving execution efficiency (e.g., reducing memory required, improving accuracy of interactions, reducing network traffic, etc.), among other options.

In some embodiments, the network 308 may include any communication network through which computer systems may exchange data. To exchange data using the network 308, the computer systems 302, 304 and 306 and the network 308 may use various methods, protocols and standards, including, among others, Fibre Channel, Token Ring, Ethernet, Wireless Ethernet, Bluetooth, IP, IPV6, TCP/IP, UDP, DTN, HTTP, FTP, SNMP, SMS, MMS, SS7, JSON, SOAP, CORBA, REST and Web Services. To ensure data transfer is secure, the computer systems 302, 304 and 306 may transmit data via the network 308 using a variety of security measures including, for example, TLS, SSL or VPN. While the distributed computer system 300 illustrates three networked computer systems, the distributed computer system 300 is not so limited and may include any number of computer systems and computing devices, networked using any medium and communication protocol.

As illustrated in FIG. 3, the computer system 302 includes a processor 310, a memory 312, a bus 314, an interface 316 and data storage 318. To implement at least some of the aspects, functions and processes disclosed herein, the processor 310 performs a series of instructions that result in manipulated data. The processor 310 may be any type of processor, multiprocessor or controller. Some exemplary processors include commercially available processors such as an Intel Xeon, Itanium, Core, Celeron, or Pentium processor, an AMD Opteron processor, a Sun UltraSPARC or IBM Power5+ processor and an IBM mainframe chip. The processor 310 is connected to other system components, including one or more memory devices 312, by the bus 314.

The memory 312 stores programs and data during operation of the computer system 302. Thus, the memory 312 may be a relatively high performance, volatile, random access memory such as a dynamic random access memory (DRAM) or static memory (SRAM). However, the memory 312 may include any device for storing data, such as a disk drive or other non-volatile storage device. Various examples may organize the memory 312 into particularized and, in some cases, unique structures to perform the functions disclosed herein. These data structures may be sized and organized to store values for particular data and types of data.

Components of the computer system 302 are coupled by an interconnection element such as the bus 314. The bus 314 may include one or more physical busses, for example, busses between components that are integrated within the same machine, but may include any communication coupling between system elements including specialized or standard computing bus technologies such as IDE, SCSI, PCI and InfiniBand. The bus 314 enables communications, such as data and instructions, to be exchanged between system components of the computer system 302.

The computer system 302 also includes one or more interface devices 316 such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system 302 to exchange information and to communicate with external entities, such as users and other systems.

The data storage 318 includes a computer readable and writeable nonvolatile, or non-transitory, data storage medium in which instructions are stored that define a program or other object that is executed by the processor 310. The data storage 318 also may include information that is recorded, on or in, the medium, and that is processed by the processor 310 during execution of the program. More specifically, the information may be stored in one or more data structures specifically configured to conserve storage space or increase data exchange performance.

The instructions stored in the date storage may be persistently stored as encoded signals, and the instructions may cause the processor 310 to perform any of the functions described herein. The medium may be, for example, optical disk, magnetic disk or flash memory, among other options. In operation, the processor 310 or some other controller causes data to be read from the nonvolatile recording medium into another memory, such as the memory 312, that allows for faster access to the information by the processor 310 than does the storage medium included in the data storage 318. The memory may be located in the data storage 318 or in the memory 312, however, the processor 310 manipulates the data within the memory, and then copies the data to the storage medium associated with the data storage 318 after processing is completed. A variety of components may manage data movement between the storage medium and other memory elements and examples are not limited to particular data management components. Further, examples are not limited to a particular memory system or data storage system.

Although the computer system 302 is shown by way of example as one type of computer system upon which various aspects and functions may be practiced, aspects and functions are not limited to being implemented on the computer system 302 as shown in FIG. 3. Various aspects and functions may be practiced on one or more computers having different architectures or components than that shown in FIG. 3. For instance, the computer system 302 may include specially programmed, special-purpose hardware, such as an application-specific integrated circuit (ASIC) tailored to perform a particular operation disclosed herein. While another example may perform the same function using a grid of several general-purpose computing devices running MAC OS System X with Motorola PowerPC processors and several specialized computing devices running proprietary hardware and operating systems.

The computer system 302 may be a computer system including an operating system that manages at least a portion of the hardware elements included in the computer system 302. In some examples, a processor or controller, such as the processor 310, executes an operating system. Examples of a particular operating system that may be executed include a Windows-based operating system, such as, Windows NT, Windows 2000 (Windows ME), Windows XP, Windows Vista, Windows 7 or 8 operating systems, available from the Microsoft Corporation, a MAC OS System X operating system available from Apple Computer, one of many Linux-based operating system distributions, for example, the Enterprise Linux operating system available from Red Hat Inc., a Solaris operating system available from Sun Microsystems, or a UNIX operating systems available from various sources. Many other operating systems may be used, and examples are not limited to any particular operating system.

The processor 310 and operating system together define a computer platform for which application programs in high-level programming languages are written. These component applications may be executable, intermediate, bytecode or interpreted code which communicates over a communication network, for example, the Internet, using a communication protocol, for example, TCP/IP. Similarly, aspects may be implemented using an object-oriented programming language, such as .Net, SmallTalk, Java, C++, Ada, C# (C-Sharp), Objective C, or Javascript. Other object-oriented programming languages may also be used. Alternatively, functional, scripting, or logical programming languages may be used.

Additionally, various aspects and functions may be implemented in a non-programmed environment, for example, documents created in HTML, XML or other format that, when viewed in a window of a browser program, can render aspects of a graphical-user interface or perform other functions. Further, various examples may be implemented as programmed or non-programmed elements, or any combination thereof. For example, a web page may be implemented using HTML while a data object called from within the web page may be written in C++. Thus, the examples are not limited to a specific programming language and any suitable programming language could be used. Accordingly, the functional components disclosed herein may include a wide variety of elements, e.g., specialized hardware, executable code, data structures or data objects, that are configured to perform the functions described herein.

In some examples, the components disclosed herein may read parameters that affect the functions performed by the components. These parameters may be physically stored in any form of suitable memory including volatile memory (such as RAM) or nonvolatile memory (such as a magnetic hard drive). In addition, the parameters may be logically stored in a propriety data structure (such as a database or file defined by a user mode application) or in a commonly shared data structure (such as an application registry that is defined by an operating system). In addition, some examples provide for both system and user interfaces that allow external entities to modify the parameters and thereby configure the behavior of the components.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

What is claimed is:

1. An execution system, the system comprising:
at least one processor operatively connected to a memory;
a modelling component, executed by the at least one processor, configured to:
automatically identify queries based on a first machine learning model;
automatically identify follow up queries responsive to the participants answers to the queries based on a second machine learning model;
a visit simulator, executed by the at least one processor, configured to:
present queries identified by the modelling component;
accept responses from the clinical trial participant;
present the follow up queries responsive to the participants answers in real time; and
display instructions for treatment administration or data collection tailored to the participants requests.

2. The system of claim 1, further comprising a compliance evaluator component, executed by the at least one processor, configured to monitor participant compliance with client trial requirements and generate predictions for non-compliance in treatment and/or collection.

3. The system of claim 1, wherein the visit simulator, executed by the at least one processor, is further configured to guide participants through use of measurement devices associated with a clinical trial execution.

4. The system of claim 3, wherein the visit simulator is configured to automatically select audio and visual prompts to the participants.

5. The system of claim 4, wherein the visit simulator is configured to:
monitor data capture from the measurement devices;
generate a capture quality metric for the monitored data; and
generate video and/or audio instruction to the participant to optimize localization, operation, or positioning of the measurement device to improve capture quality.

6. The system of claim 4, wherein the visit simulator is configured to:
monitor video capture of the participant during use of the measurement devices;
generate video and/or audio instruction to the participant to optimize localization, operation, or positioning of the measurement device to improve capture quality.

7. The system of claim 5, wherein instruction occurs in real or near real time.

8. The system of claim 5, wherein the system is configured to generate an optimal data capture model for each respective participants, and generate tailored instructions to the respective participants based on identified positioning, localization, or operation for best data capture results.

9. A clinical trial execution system, the system comprising:
at least one processor operatively connected to a memory;
a protocol analyzer component, executed by the at least one processor, configured to:
define protocol requirements to be performed by a plurality of clinical trial participants;
validate pre-requisites for performing steps of the clinical trial protocol;
define boundaries for time ranges to execute a virtual visit with a participant that meet at least a minimum temporal requirement defined by the protocol requirements;
a visit simulator, executed by the at least one processor, configured to:
present queries identified as at least part of the protocol requirements;
accept responses from the clinical trial participant;
display instructions for treatment administration or data collection based at least in part on the protocol requirements; and
capture health or device data associated with the protocol requirements to be performed by the plurality of clinical trial participants.

10. The system of claim 9, wherein the visit simulator is configured to generate and present follow up queries responsive to the participants answers in real time, wherein the follow up queries are tailored to the participants requests.

11. The system of claim 9, further comprising a compliance evaluator component, executed by the at least one processor, configured to monitor participant compliance with client trial requirements.

12. The system of claim 9, wherein the visit simulator, executed by the at least one processor, is further configured to guide participants through use of measurement devices associated with a clinical trial execution.

13. The system of claim 12, wherein the visit simulator is configured to automatically select audio and visual prompts to the participants.

14. The system of claim 13, wherein the visit simulator is configured to:
monitor data capture from the measurement devices;
generate a capture quality metric for the monitored data; and
generate video and/or audio instruction to the participant to optimize localization, operation, or positioning of the measurement device to improve capture quality.

15. The system of claim 13, wherein the visit simulator is configured to:
monitor video capture of the participant during use of the measurement devices;
generate video and/or audio instruction to the participant to optimize localization, operation, or positioning of the measurement device to improve capture quality.

16. A computer based method for clinical trial execution, the method comprising:
defining, by at least one processor, protocol requirements to be performed by a plurality of clinical trial participants;
validating, by the at least one processor, pre-requisites for performing steps of the clinical trial protocol;
defining, by the at least one processor, boundaries for time ranges to execute a virtual visit with a participant that meet at least a minimum temporal requirement defined by the protocol requirements;
presenting, by the at least one processor, queries identified as at least part of the protocol requirements;
accepting, by the at least one processor, responses from the clinical trial participant;
displaying, by the at least one processor, instructions for treatment administration or data collection based at least in part on the protocol requirements; and
capturing, by the at least one processor, health or device data associated with the protocol requirements to be performed by the plurality of clinical trial participants.

17. The method of claim 16, wherein the method further comprises generating and presenting follow up queries responsive to the participants answers in real time, wherein the follow up queries are tailored to the participants requests.

18. The method of claim 16, wherein the method further comprises monitoring participant compliance with client trial requirements.

19. The method of claim 16, wherein the method further comprises automatically guiding participants through use of measurement devices associated with a clinical trial execution.

20. The method of claim 16, wherein the method further comprises
monitoring data capture from the measurement devices;
generating a capture quality metric for the monitored data; and
generating video and/or audio instruction to the participant to optimize localization, operation, or positioning of the measurement device to improve capture quality.
